(19) United States
(12) Patent Application Publication
(10) Pub. No.: US 2008/0304623 A1
(43) Pub. Date: Dec. 11, 2008

(54) CONTROL UNIT AND METHOD FOR CONTROLLING A RADIATION THERAPY SYSTEM, AND RADIATION THERAPY SYSTEM

(76) Inventor: Eike Rietzel, Darmstadt (DE)

Correspondence Address: BRINKS HOFER GILSON & LIONE, P.O. BOX 10395, CHICAGO, IL, 60610, US

(21) Appl. No.: 12/132,447

(22) Filed: Jun. 3, 2008

(30) Foreign Application Priority Data
Jun. 8, 2007 (DE) 10 2007 026 516.8

(51) Int. Cl. A61B 6/00

(52) U.S. Cl. 378/65

(57) ABSTRACT

A control system for controlling a radiation therapy system is provided. The control system includes a sensor operable to measure a pressure in the interior of a body; and a control unit operable to output a control signal for a beam interruption unit as a function of a pressure status in the interior of the body.

The present patent document claims the benefit of the filing date of DE 10 2007 026 516.8, filed Jun. 8, 2007, which is hereby incorporated by reference.

### BACKGROUND

The present embodiments relate controlling a radiation therapy system.

Radiation therapy is used to treat diseases, such as tumors. During radiation therapy, radiation beams are aimed at a target volume (tissue), which is to be irradiated, of a body. During particle therapy, protons, carbon ions, or other ions, for example, are accelerated to high energy levels and aimed at the tissue to be irradiated. Other types of beams, such as electron or X-ray beams, can be used for the radiation treatment. The interaction of the treatment beam with the tissue causes the death of the tissue irradiated.

To irradiate a target volume as precisely as possible, the treatment beam outputs energy as much as possible in the target volume. The surrounding tissue is spared from the treatment beam output energy. The target volume may not strike the tissue as planned because of movements of the tissue to be irradiated. Some of the energy of the treatment beam, because of the motion, can be output in the tissue that is supposed to be spared. Accordingly, the quality of the radiation treatment may be reduced and side effects may be increased.

Imaging may be done directly before the radiation treatment to detect the internal anatomy of the patient. At the onset of a radiation treatment session, the volume to be irradiated is positioned as precisely as possible and is struck as precisely as possible by the treatment beam. Based on the imaging, the position of the patient and/or radiation treatment parameters can be adapted to the situation ascertained by the imaging (e.g., adaptive radiation therapy (ART)).

During the radiation treatment procedure, movement can occur in the target volume that is to be irradiated. Efforts have been directed to detecting the movements during a radiation treatment procedure and adapting the irradiation during the radiation treatment procedure accordingly.

US Patent Disclosure 2003/0136924 A1 discloses a device and a method in which the movement during a radiation treatment procedure is monitored with the aid of optical cameras and/or X-ray systems, and an ion beam is adapted to the movement during the radiation treatment procedure.

Calypso Medical Technologies Inc. offers a system with high-frequency markers that can be implanted in a prostate. A deviation in the target volume from a planned irradiation position can be detected.

### SUMMARY AND DESCRIPTION

The present embodiments may obviate one or more drawbacks or limitations inherent in the related art. For example, in one embodiment, a radiation therapy system may be controlled, such that a moving target volume can be irradiated in a simple way, safely and reliably and with few side effects.

In one embodiment, a control unit for controlling a radiation therapy system includes a sensor, with which a pressure in the interior of a body is measurable; and a unit for outputting a control signal for a beam interruption unit as a function of a pressure status in the interior of the body. The radiation therapy system may be used to irradiate a target volume.

Pressure fluctuations may occur in the interior of a body. The pressure fluctuations may cause a shift in the position of the target volume. By measuring these pressure fluctuations, improved control of a radiation treatment procedure is possible. The irradiation may be interrupted as a function of the measured pressure. Whenever a shift in the position of the target volume is likely because of a pressure fluctuation, the irradiation is interrupted. Movements in the interior of the body during the radiation treatment may be taken into account without having to implant expensive markers or having to perform an additional scan of the body. A change in position of the target volume to be irradiated, caused by pressure fluctuations in the interior of the body, does not lead to an incorrect irradiation of the target volume.

The change in position of the target volume may be reversible, as soon as the pressure has normalized. A radiation treatment may be resumed when normalizing of the measured pressure is detected by the sensor.

In one embodiment, the sensor may measure pressure in a hollow organ of the body. Pressure fluctuations in organs that are subject to frequent pressure fluctuations, namely hollow organs, may be measured. The pressure may be measured in a non-hollow organ; for example, the pressure may be measured inside the abdominal wall or inside other tissue and organs that are not hollow.

In one embodiment, the sensor for the pressure measurement is in the intestine. Pressure fluctuations that are due to intestinal gases or movement of intestinal gas masses can be measured. Radiation treatment procedures that are performed in the region of the intestine may be improved. Radiation treatment procedures that performed in the region of the intestine may include, for example, radiation treatments of the prostate, in the lower abdomen, and in the abdomen. The pressure measurement may take place in other hollow organs, such as the bladder, the lungs, the stomach, or other hollow organs.

In one embodiment, the sensor may be introduced rectally into the intestine, which may provide an easy access route that allows unproblematic placement of the sensor in a patient.

In one embodiment, the sensor measures gas pressure. Pressure fluctuations in the intestine that are due to intestinal gas masses, for example, may be detected.

The sensor may be mounted in a system having a valve mechanism that is used for letting off (releasing) the overpressure. For example, via the valve mechanism, an overpressure occurring in the intestine may be let off (released) rectally. The pressure conditions in the intestine normalize faster, so that a continuation of the radiation treatment procedure may be resumed faster. The valve may remain open until the overpressure in the intestine has been let off (released).

The sensor may be disposed on an inflatable balloon. An inflatable rectal balloon, for example, may reinforce the immobilization of organs, such as the prostate.

A control unit may include an evaluation unit, with which a signal generated by the sensor is evaluated. The control signal may be output to a beam interruption unit as a function of an evaluation of the evaluation unit. With the evaluation unit, complex algorithms for evaluation may be implemented and adapted.

In one embodiment, a temporary beam disruption may be induced, for example, whenever the evaluation unit ascertains an evaluation of the sensor signal from a tolerance range or an exceeding of a threshold value.

A radiation therapy system for irradiating a target volume to be irradiated in a body may include a beam interruption unit and a control unit. In another embodiment the radiation therapy system may be a particle therapy system. However, it is also possible for the radiation therapy system to be a conventional beam therapy unit, which, for example, is used with a LINAC for generating high-energy X-rays or electron beams.

A method for controlling a radiation therapy system may include measuring a pressure in the interior of a body with a sensor; and interrupting a radiation treatment procedure as a function of a pressure status in the interior of the body.

The method may be used to attain a change in position of the target volume to be irradiated, caused by pressure fluctuations in the interior of the body, does not cause incorrect irradiation of the target volume.

The pressure may be measured in a hollow organ of the body, such as in an intestine, and the sensor is introduced into the intestine, for example, rectally. As the pressure in the interior of the body, a gas pressure may, for example, be measured.

A signal generated by the sensor is evaluated, and the radiation treatment procedure is interrupted as a function of an evaluation of the sensor signal. For example, the radiation treatment procedure may be interrupted temporarily whenever a deviation in the sensor signal from a tolerance range or an exceeding of a threshold value is ascertained.

### BRIEF DESCRIPTION OF THE DRAWINGS

FIG. 1 illustrates one embodiment of a particle therapy system;

FIG. 2 illustrates a sagittal section through a human body;

FIG. 3 illustrates one embodiment of a radiation treatment procedure; and

FIG. 4 illustrates one embodiment of a method for controlling a radiation therapy system.

### DETAILED DESCRIPTION

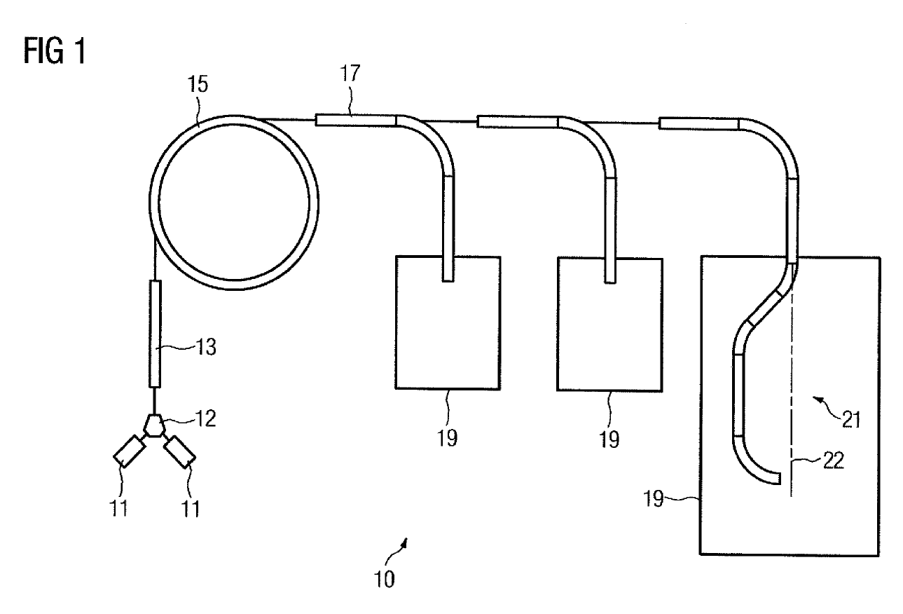

FIG. 1 shows a particle therapy system 10. The particle therapy system 10 may be used to irradiate a body, such as tissue diseased by tumor, with a particle beam.

The particle beam may include ions, such as protons, pions, helium ions, carbon ions, or other types of ions. The particles may be generated in a particle source 11. As shown in FIG. 1, the system 10 may include two particle sources 11 that generate two different types of ions. It is possible to switch rapidly between the two types of ions. A switching magnet 12 may, for example, be used to switch rapidly between the two types of ions. The switching magnet 12 may be disposed between the ion sources 11 and a preaccelerator 13. As an example, the particle therapy system 10 may be operated with protons and carbon ions simultaneously.

The ions generated by the ion sources or one of the ion sources 11 and optionally selected using the switching magnet 12 may be accelerated to a first energy level in the preaccelerator 13. The preaccelerator 13 may, for example, be a linear accelerator (LINAC, for LINear ACcelerator). The particles may be fed into an accelerator 15, such as a synchrotron or cyclotron. In the accelerator 15, they are accelerated to radiation treatment energy levels used for the radiation treatment. Once the particles leave the accelerator 15, a high-energy-beam transport system 17 carries the particle beam to one or more radiation treatment rooms or chambers 19. In a radiation treatment room or chamber 19, the particles that have been accelerated are aimed at a body that is to be irradiated. Depending on the design, this is done from a fixed direction (e.g., in a “fixed-beam” rooms or chambers), or from various directions via a rotatable gantry 21 that is movable about an axis 22 (e.g., in a “gantry-based” room).

A particle therapy system 10, as shown in FIG. 1, may include a particle therapy system, but it can also deviate from the particle therapy system. The exemplary embodiments described below may be used both in conjunction with the particle therapy system shown in FIG. 1 and with other particle therapy systems or radiation therapy systems or equipment.

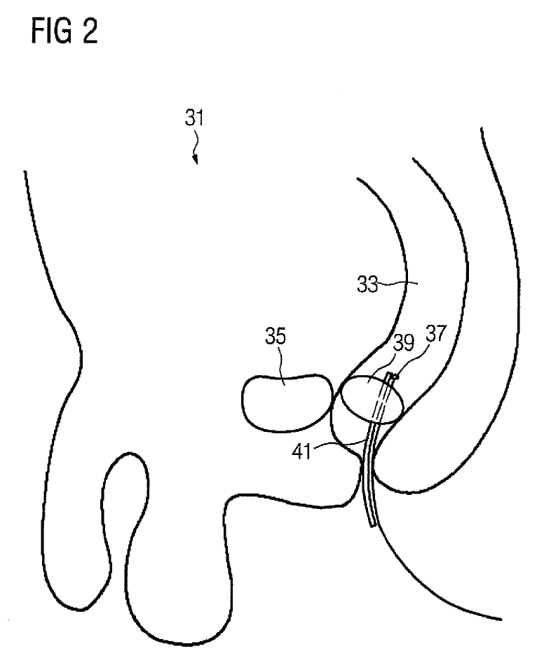

FIG. 2 shows a schematic sagittal section 31 through the lower abdomen of a male body. The outer contours and the contours of the organs that are used for the ensuing explanation are shown. In the lower abdomen, near the rectum 33, is the prostate 35. The prostate 35 is an organ whose tumors are often treated by radiation therapy. During a radiation treatment, the prostate 35 may move inside the body, or may cause internal movements. The movements are due predominantly to gas production and/or shifting of gas masses in the adjacent intestine or the adjacent rectum 33.

A sensor 37, with which the pressure in the interior of the intestine may be measured, is introduced rectally into the rectum 33. The sensor 37, in this exemplary embodiment, is disposed on a rectal balloon 39, which may be used to immobilize the prostate 35. The rectal balloon 39 may be introduced into the rectum 33 in a folded-up state. By inflation of the rectal balloon 33, a mechanical fixation of the prostate 35 up to a certain degree may be attained.

Despite the immobilization, it is possible that a pressure increase in the rectum 33 or in the intestine, caused by gas masses, may cause the location of the prostate 35 to shift. The sensor 37 may detect the pressure fluctuations. Based on the pressure fluctuations, the radiation treatment procedure may be interrupted.

The rectal balloon 39 may have a valve 41. The valve 41 may be used so that intestinal gas masses, in the event of a pressure increase, may escape. A valve 41 opens as soon as the intestinal pressure has reached a threshold value, and it may remain open until such time as the gas masses have escaped enough that the rectal or intestinal overpressure has normalized again. The release of gas may compensate for an overpressure occurring in the intestine. The release of gas may cause a return of the organ to be irradiated to its original position, so that an interrupted radiation treatment may be resumed.

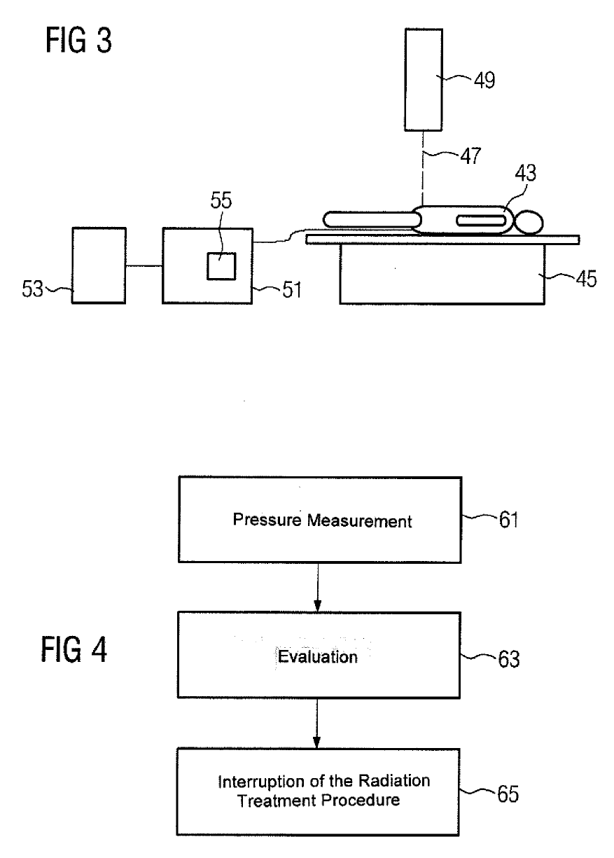

FIG. 3 shows a patient 43, who is positioned in a treatment room or chamber on a treatment table 45 in such a way that the region in the lower abdomen to be irradiated is struck by a particle beam 47 that is aimed at the patient from a beam outlet 49. The sensor, which is introduced into the intestine of the patient, may record a sensor signal that characterizes the pressure conditions in the intestine.

The sensor may be carried to a therapy control unit 51. In a simple case, the therapy control unit 51 generates the output of a control signal for a beam interruption unit 53, as soon as the measured pressure exceeds a critical threshold value. Alternatively, the control signal for the beam interruption unit 53 may be output whenever the measured pressure is outside a tolerance range that, for example, is predetermined.

The interruption in the radiation treatment procedure may be controlled, for example, in such a way that the radiation treatment procedure is interrupted as long as the control signal for the beam interruption unit 53 is presented.

In one embodiment, the sensor signal may be carried to an evaluation unit 55, which is implemented, for example, in the therapy control unit 51 and in which algorithms for processing and evaluating the control signal may be implemented and adapted in a simple way.

The individual units, such as the beam interruption unit 53, the unit for outputting a control signal for the beam interruption unit, or the therapy control unit 51, and the evaluation unit 55, may be variously implemented in a particle therapy system, for example, either as separate units or together in a single computer unit.

FIG. 4 shows a flow chart of a method for controlling a radiation therapy system.

In a first act 61, the pressure in the interior of a body is measured, for example, in the interior of the intestine. In a second, optional act 63, a sensor signal, which characterizes the pressure in the interior of the intestine, is evaluated, for example, by an evaluation unit. In a third act 65, the interruption of the radiation treatment procedure is effected as a function of the pressure in the interior of the intestine, for example, with the aid of a control signal for a beam interruption unit.

Various embodiments described herein can be used alone or in combination with one another. The forgoing detailed description has described only a few of the many possible implementations of the present invention. For this reason, this detailed description is intended by way of illustration, and not by way of limitation. It is only the following claims, including all equivalents that are intended to define the scope of this invention.

1. A control system for controlling a radiation therapy system that is operable to irradiate a target volume, the system including:
a sensor operable to measure a pressure in the interior of a body;
a control unit operable to output a control signal for a beam interruption unit as a function of a pressure status in the interior of the body.

2. The control system as defined by claim 1, wherein the sensor measures pressure in a hollow organ of the body.

3. The control system as defined by claim 2, wherein the sensor measures pressure in an intestine.

4. The control system as defined by claim 3, wherein the sensor is introduced rectally into the intestine.

5. The control system as defined by claim 1, wherein the sensor measures a gas pressure.

6. The control system as defined by claim 1, wherein the sensor is disposed on a valve mechanism for releasing an overpressure.

7. The control system as defined by claim 1, wherein the sensor is disposed on an inflatable balloon.

8. The control system as defined by claim 1, further comprising: an evaluation unit operable to evaluate a sensor signal generated by the sensor, wherein the control signal for the beam interruption unit is output as a function of an evaluation of the evaluation unit.

9. The control system as defined by claim 8, wherein the control signal for the beam interruption unit is operable to disrupt the beam when the sensor signal deviates from a tolerance range or exceeds a threshold value.

10. A radiation therapy system for irradiating a target volume, the system comprising:
a sensor operable to measure a pressure in the interior of a body;
a control unit operable to interrupt a radiation beam as a function of the pressure in the interior of the body.

11. The radiation therapy system as defined by claim 10, wherein the radiation beam is a particle beam.

12. A method for controlling a radiation therapy system, the method including:
measuring a pressure in the interior of a body with a sensor;
interrupting a radiation treatment procedure as a function of the pressure in the interior of the body.

13. The method as defined by claim 12, wherein measuring a pressure includes measuring a pressure in a hollow organ of the body.

14. The method as defined by claim 13, wherein measuring a pressure in a hollow organ of the body includes measuring a pressure in an intestine.

15. The method as defined by claim 12, wherein measuring a pressure in the interior of the body includes measuring a gas pressure in the interior of a body.

16. The method as defined by claim 12, comprising: evaluating a signal generated by the sensor, and interrupting the radiation treatment procedure as a function of an evaluation of the sensor signal.

17. The method as defined by claim 12, wherein the radiation treatment procedure is interrupted temporarily whenever a deviation in the sensor signal from a tolerance range or an exceeding of a threshold value is ascertained.
